(19) United States
(12) Plant Patent Application Publication
(10) Pub. No.: US 2015/0173285 P1
(43) Pub. Date: Jun. 18, 2015

(54) Poinsettia plant named 'NPCW13218'

(50) ;

(71) Applicant: Klemm+Sohn GmbH & Co. KG, Stuttgart (DE)

(72) Inventors: Nils Klemm, Stuttgart (DE); Guido von Tubeuf, Stuttgart (DE)

(73) Assignee: Klemm+Sohn GmbH & Co. KG, Stuttgart (DE)

(21) Appl. No.: 13/998,917

(22) Filed: Dec. 18, 2013

(51) Int. Cl. A01H 5/00

(52) U.S. Cl. PLT/307

(57) ABSTRACT

A new poinsettia plant particularly distinguished by having bright red bracts, a triangular plant form, and an upright growth habit, is disclosed.

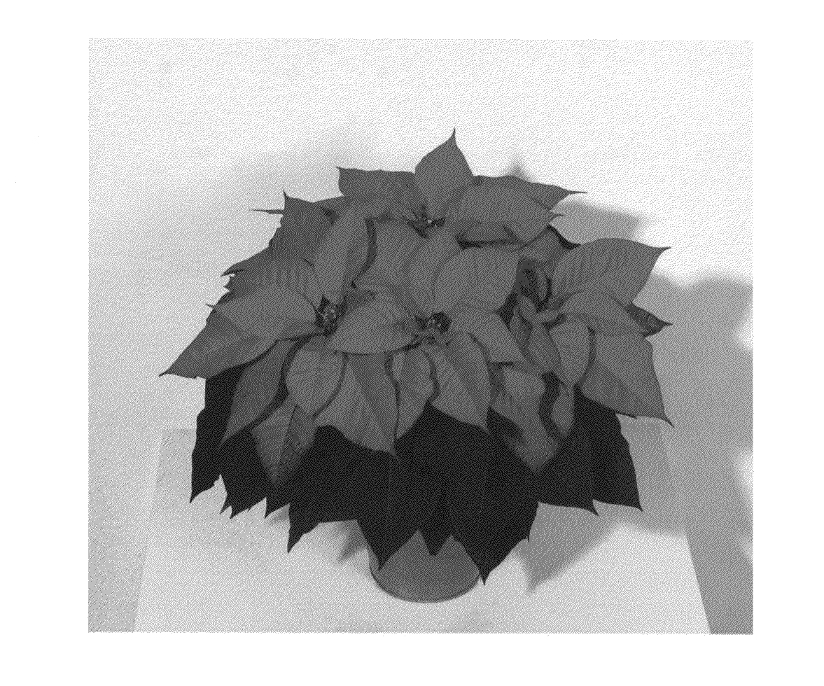

### GENUS AND SPECIES

Euphorbia pulcherrima

### VARIETY DENOMINATION

‘NPCW13218’

### BACKGROUND OF THE NEW PLANT

The present invention comprises a new and distinct variety of poinsettia, botanically known as Euphorbia pulcherrima and hereinafter referred to by the variety name ‘NPCW13218’. ‘NPCW13218’ was derived as a selection of a single, bright red plant among a population derived from irradiated cuttings of the poinsettia ‘NPCW10164’ (U.S. Plant Pat. No. 22,597) and commercially known as ‘Christmas Day’, having red bracts.

In November 2010, a single bright red plant was selected in Stuttgart, Germany from irradiated lines of poinsettia ‘NPCW10164’. In February 2011, ‘NPCW13218’ was first asexually propagated by vegetative cuttings. ‘NPCW13218’ was found to reproduce true to type in successive generations of asexual propagation via vegetative cuttings in Stuttgart, Germany.

### CROSS REFERENCE TO RELATED APPLICATIONS

Plant Breeder's Rights for ‘NPCW13218’ were applied for in Europe on Dec. 9, 2013, however an application number has not been assigned. ‘NPCW13218’ has not been made publicly available or sold more than one year prior to the filing date of this application.

### SUMMARY

The following are the most outstanding and distinguishing characteristics of this new variety when grown under normal horticultural practices in Stuttgart, Germany.

1. Bright red bracts;

2. A triangular plant form; and

3. An upright growth habit.

### DESCRIPTION OF THE PHOTOGRAPH

This new poinsettia plant is illustrated by the accompanying photograph which shows an overall view of the plant. The photograph is of a plant approximately four-months-old grown indoors in Stuttgart, Germany in November 2013. The colors shown are as true as can be reasonably obtained by conventional photographic procedures.

### DESCRIPTION OF THE NEW VARIETY

The following detailed descriptions set forth the distinctive characteristics of ‘NPCW13218’. The data which define these characteristics were collected from asexual reproductions carried out in Stuttgart, Germany. Data was collected on four-month-old plants grown indoors in Stuttgart, Germany under greenhouse conditions in the fall of 2013. Color references are to The R.H.S. Colour Chart of The Royal Horticultural Society of London (R.H.S.), 5^{th} edition (2007).
Classification:
   Family.—Euphorbiaceae.
   Species.—Euphorbia pulcherrima.
   Common name.—Poinsettia.
   Variety.—‘NPCW13218’.
Parentage: Selection from the irradiated Poinsettia ‘NPCW10164’
Growth:
   Time to produce a rooted cutting.—4 weeks.
   Blooming habit.—Intermittent.
   Flowering response time.—Approximately 53 days.
   Keeping quality.—Very good.
Plant description:
   Form.—Triangular.
   Growth habit.—Upright.
   Height.—28.0 cm to 30.0 cm.
   Width.—25.0 cm to 30.0 cm.
   Number of branches.—5 to 6.
   Average number of inflorescences per plant.—5.5.
Propagation:
   Type cuttings.—Apical cuttings.
   Time to produce a rooted cutting.—4 weeks.
Stems:
   Color.—RHS 143A.
   Length.—25.0 cm.
   Internode length.—1.2 cm to 3.5 cm.
   Diameter.—0.5 cm to 0.8 cm.
Leaves:
   Quantity.—6 to 7 per lateral branch.
   Arrangement.—Alternate.
   Shape.—Ovate.
   Apex.—Acute.
   Base.—Obtuse.
   Margin.—Serrate.
   Length.—8.0 cm to 14.0 cm.
   Width.—6.0 cm to 9.0 cm.
   Lobes.—Lobation characteristics: Moderately deep Texture (both surfaces): Smooth Vein color: Upper surface: Transparent Lower surface: RHS 139D; middle vein is slightly reddish.
   Variegation.—Absent.
   Color.—Mature foliage: Upper surface: RHS 139A Lower surface: RHS 139D Immature foliage: Upper surface: RHS 141A Lower surface: RHS 139B.
   Leaf petiole.—Length: 3.0 cm to 6.0 cm Diameter: 0.2 cm to 0.3 cm Color: Upper (part) surface: RHS 59A Lower (part) surface: RHS 181B.
Inflorescence:
   Lastingness of inflorescence on the plant.—4 weeks.
   Diameter.—27.0 cm to 30.0 cm.
   Height.—3.0 cm to 4.0 cm.
   Fragrance.—Absent.
Bracts:
   Number of bracts per inflorescence.—6 to 9.
   Shape.—Ovate.
   Apex.—Acute.
   Base.—Obtuse.
   Margin.—Mostly entire but older bracts are slightly senate.
   Size.—Length: 9.0 cm to 11.0 cm Width: 4.0 cm to 8.0 cm.
   Texture (both upper and lower surfaces).—Smooth.
   Vein color.—Upper surface: RHS 59A Lower surface: RHS 181B.
   Bract color.—Upper surface: RHS 46B Lower surface: RHS 47B.
   Bract petiole.—Length: 0.5 cm to 2.5 cm Diameter: 0.2 cm to 0.3 cm Color: Upper (part) surface: RHS 59A Lower (part) surface: RHS 181B.
Cyme:
   Diameter.—1.0 cm to 2.5 cm.
   Cyathia number.—7 to 10 per inflorescence.
   Cyathium.—Shape: Ovate Diameter: 0.2 cm to 0.6 cm Length: 0.3 cm to 0.8 cm Color: RHS 143C.
   Peduncle.—Color: RHS 142B Length: 0.2 cm to 0.3 cm.
   Nectar cups.—Number: 1 per cyathium Diameter: 0.1 cm Size: 0.4 cm to 0.5 cm Color: RHS 1C.
Reproductive organs:
   Stamen quantity.—5 to 10 per cyanthium.
   Stamen shape.—Ovate.
   Filament length.—0.25 cm.
   Filament color.—RHS 46A.
   Pollen quantity.—Moderate.
   Pollen color.—RHS 12A.
   Gynoecium.—Present. Pistil quantity per cyathium: 1 Pistil length: 0.2 cm, Stigma form: Divided in 2 parts Stigma color: RHS 183A Style length 0.2 cm Style color: HS 183A Ovary length: 0.05 cm Ovary color: RHS 143C.
Fruit and seed set: None observed.

### COMPARISON WITH PARENTAL LINE AND KNOWN VARIETY

‘NPCW13218’ is a distinct variety of Poinsettia having bright red bracts. ‘NPCW13218’ is distinguished from its parent ‘NPCW10164’ as described in Table 1.

TABLE 1

| | Variety | Parent |
|---|---|---|
| Characteristic | ‘NPCW13218’ | ‘NPCW10164’ |
| Bract color, upper surface | RHS 46B | RHS 46A |
| Bract color, lower surface | RHS 47B | RHS 47A |
| Leaf quantity per lateral branch | 6 to 7 | 8 to 15 |

‘NPCW13218’ can be compared to the commercial Poinsettia plant variety ‘NPCW06115’ (U.S. Plant Pat. No. 19,020). Differences between the two varieties are described in Table 2.

TABLE 2
Comparison with Similar Variety

| | | Commercial line |
|---|---|---|
| Characteristic | ‘NPCW13218’ | ‘NPCW06115’ |
| Plant width or spread | 25.0 cm to 30.0 cm | 30.0 cm to 35.0 cm |
| Inflorescence diameter | 27.0 cm to 30.0 cm | 15.0 cm to 20.0 cm |

We claim:

1. A new and distinct variety of poinsettia plant named ‘NPCW13218’ as shown and described herein.
